(19) United States
(12) Patent Application Publication
(10) Pub. No.: US 2008/0249715 A9
(43) Pub. Date: Oct. 9, 2008

(54) Method for identifying markers

(76) Inventor: Theodore E. Maione, Wakefield, MA (US)

Correspondence Address: SILLS CUMMIS & GROSS P.C., ONE ROCKEFELLER PLAZA, IP DEPARTMENT - 25TH FLOOR, NEW YORK, NY, 10020, US

(21) Appl. No.: 10/859,755

(22) Filed: Jun. 3, 2004

Related U.S. Application Data
(63) Continuation of application No. PCT/US02/38463, filed on Dec. 3, 2002.
(60) Provisional application No. 60/334,606, filed on Dec. 3, 2001.

(51) Int. Cl. G06F 19/00; G01N 33/48; G01N 33/50

(52) U.S. Cl. 702/19

(57) ABSTRACT

The invention is a method for identifying markers associated with the presence of a predetermined characteristic comprising accumulating spectral data from a sample known to have a predetermined characteristic, identifying a spectral feature which is indicative of the predetermined characteristic, and identifying a marker associated with the spectral feature.

### CROSS REFERENCE TO RELATED APPLICATIONS

The present application claims benefit of U.S. Provisional Patent Ser. No. 60/334,606 filed Dec. 3, 2001, and application Serial No. U.S. 02/38463 filed under the Patent Cooperation Treaty Dec. 3, 2002.

### FIELD OF THE INVENTION

The present invention relates to methods for identifying markers associated with the presence of a known characteristic in a sample. In particular, the markers are fluorescent molecules indicative of the presence of a disease or involved in the disease process.

### BACKGROUND OF THE INVENTION

In addition to genes and proteins, there exist categories of biological molecules, including carbohydrates and small organic molecules that hold major physiological significance. The variations in these molecules represent the complex interaction of the organism's genome and proteome with environmental factors that include diseases. Alterations in a subject's profile may have linkage to an acute disease or correlations with disease progression.

Establishing the relationship between the profile of small organic molecules and specific diseases provides another pathway for building new approaches to early diagnosis and treatment of infectious, cancerous, and metabolic diseases. Perhaps of greatest importance is the potential power of this approach for the prospective detection of indicators of ‘subclinical’ disease in healthy individuals and development of individualized disease prevention strategies. The analysis of these non-genetic molecules seeks to correlate the effects of the broadest range of environmental influences (i.e., infectious agents, diet, exposure to toxins) on the complete portfolio of biological molecules found in an organism over time.

The invention exploits the high sensitivity and information content of natural fluorescence (or intrinsic fluorescence) as its primary approach to establishing disease spectral profiles. The natural portfolio of fluorescent molecules present in human cells and fluids include many structurally diverse molecular families with widely divergent biologic roles. The array of intrinsically fluorescent molecules that a host possesses therefore represents a broad view of the physiological status of the organism. Furthermore, aberrations of any biochemical pathway are likely to ultimately lead to a disruption of the normal physiologic level of one or more of these natural fluoresce. As markers of a disease state, these intrinsically fluorescent molecules hold great value as vehicles both to screen for disease directly and for diagnostic and therapeutic development.

### SUMMARY OF THE INVENTION

Spectra Molecular Informatics (SMI) is a method for identifying associations between specific molecules and specific diseases. By applying SMI, a disease-related discriminatory spectral signal may be identified. The method preferably monitors intrinsic fluorescence for identification of the disease-related signal, although absorbance, phosphorescence, Raman spectroscopy, extrinsic fluorescence, chemically altered intrinsic fluorescence, or other optical signals could be exploited for these purposes.

The method of the invention preferably includes accumulating spectral data, which is preferably multidimensional. The data preferably include intrinsic fluorescence arising from samples having a known disease state. The method further includes the identification of spectra signals, which are preferably indicative of the presence of disease, more preferably indicative of the presence of a specific disease. The identification step preferably includes subjecting the spectral to one or more data reduction steps. Based upon the spectral signals, preferably the spectral signals that are indicative of a specific disease, compounds associated with the appearance of the spectral signals are identified, preferably to at least the extent that some structural or physio-chemical properties useful for determining the presence of the molecule in a sample are characterized. For example, the spectral signals may be used in a separative technique such as chromatography to identify and preferably isolate the molecules carrying the discriminatory signal.

In one embodiment, the identified markers are used to determine the presence of the disease based upon detection of the presence of the molecule in a sample acquired from an individual.

In another embodiment the identified molecules are used to discover drugs or other treatment modalities useful in treating the disease. For example, in one embodiment a chemical library is searched for substances that interact with the identified molecule or modulate the activity of the molecule. By providing identified molecules that are known to be associated with a particular disease the present invention provides a disease-focused strategy to funding new drugs.

In another embodiment the identification of molecules are used to identify biochemical pathways, such as enzyme pathways, exhibiting aberrant behavior, such as up or down regulation, associated with the disease. This may include identification of precursor compounds associated with the disease.

In one embodiment, the present invention relates to the identification of intrinsically fluorescent molecules as indicators of the presence of disease. This category of molecules typically includes smaller organic molecules containing, for example, well-defined ring structures and/or several complex bond structures. These fluorescently active biomolecules are important indicators of disrupted physiology in virtually any disease. The molecules identified by the invention are preferably distinct from the DNA and proteins that are the subject of Genomics and Proteomics respectively, and thus represent an import pathway to obtaining disease-specific information. Thus present invention address problems that are not easily approached by genomic or proteonomic methods.

In another embodiment, the Spectra-Molecular Informatics of the invention is applied to any disease where additional diagnostic markers and therapeutic targets would have particular clinical value. The method includes the parallel identification of new markers for cancer types, neurological diseases, heart disease, and other selected conditions.

Yet another aspect of the invention relates to industries such as veterinary science, food and beverage quality control, chemical contaminant analysis, and the characterization and detection of biological and chemical weapons in which the informatics method of the invention is used to obtain data indicative of, for example, the purity or quality of a particular material.

### DETAILED DESCRIPTION

Animal and human plasma, serum, urine, cerebral spinal fluid and other biological fluids are complex mixtures of proteins, lipids and metabolites representing the immunologic, hormonal, metabolic and nutritional status of individuals. This mixture includes molecules that are intrinsically fluorescent (i.e., can be excited to emit a spectrum of light without any added reagents). Due to the complex nature of these biological fluids, disease-specific fluorescent signatures may be partly or completely obscured by signals common to all individuals, and methods to enhance differential signals are commonly employed. Fluorescent molecules (fluorophores) in plasma exist both bound to proteins or free in solution. The invention uses plasma extraction methods to permit analysis of both bound and free fluorphores and a spectral database for these preparations from normal and disease-infected individuals.

In the invention, these sample preparation tools have been coupled to comprehensive spectral surveys in which the intrinsic fluorescence is analyzed over a range of excitation and emission wavelengths. The invention includes a spectral library from complementary preparation methods, which yields a high-resolution view of the fluorescent signature of a biological fluid sample.

The invention utilizes algorithms to analyze the spectral database. This process includes: 1) the building of mathematical models of fluorescence spectra from normal and infected individuals, 2) the objective testing of each model, and 3) the iterative modification of these models based upon the inclusion test sample spectra and reoptimization. This procedure is initiated by the extraction of spectral features from normal and infected fluid by multivariate statistical methods, including Principal and Independent Component Analysis, to identify the major parameters of the spectra that carry disease discrimination. These parameters become the components of linear and non-linear mathematical ‘discriminators’ functions, which are models of disease-specific spectral differences.

The fluorescence obtained from the fluid sample represents the aggregate spectra of many fluorescent molecules. The isolation and characterization of the specific molecules that give rise to fluorescence-based discrimination is an important complement to the spectral discrimination for both the development of molecule-directed diagnostics and therapeutics. The present invention exploits discriminatory spectral information to define appropriate conditions for molecular isolation from the effective sample preparation methods. This effort identifies the discriminatory molecules in effective fluorescent assays and provides the critical molecular components.

The method of the present invention comprises:
1. Spectral data accumulation;
2. Identification of disease-specific spectral signals; and
3. Identification of molecules carrying disease-specific spectral properties.

Particular embodiments of the invention may also include:
4. Development of prototype fluorescent assay for target molecule and validation of the molecular target; and
5. Market assay development.

The invention includes sample preparation methods to selectively amplify the signal from different classes of molecules. The sample preparation methods include, but not limited to, dilutions with varying formulations (e.g. pH, salts, buffers), acid/base extractions, organic solvent extractions (including 1, 2 and 3 phase systems), temperature induced fractionation, size fractionation (by filtration, chromatography, ultracentrifugation, etc.), super-critical fluid extraction, differential extraction by chemical modification coupled to any of these methods, centrifugation or filtration coupled to any of these methods, and other known methods. A matrix of preparative methods and excitation wavelengths constitutes the Standard Spectral Survey and represents a level of database complexity necessary for comparative spectral testing between normal and diseased subjects. The excitation wavelengths that can be utilized by the invention is only limited by the irradiating sources available. The wavelengths most prevalent commercially today range from 190-1200 nm.

Based on the results of the Standard Spectral Survey phase, the invention utilizes methods with enhanced selectivity and spectral resolution in a subsequent Advanced Spectral Profile phase. These methods include, but are not limited to, for fluorescence: fine emission wavelength selection, focused spectral data collection, increased radiation power; or other methods including: infrared spectroscopy, Raman spectroscopy, dual photo fluorescence, phosphorescence, X-ray fluorescence.

The invention subjects the compiled data of both the Standard Spectral Survey and Advanced Spectral Profile to multiple analytical strategies to identify spectral patterns and further characterize spectral differences between normal and disease subjects. These analytical strategies include, but are not limited to, simple processing (subtractions, normalization, user-specified computations), mathematical transformations of data (e.g. first, second, third and fourth derivatives, Fourier transformation), Principal Component Analysis (PCA) and Independent Component Analysis (ICA). Some of the spectral comparative computational methods employ multivariate analytical strategies that isolate key differentiating features of the spectra. Comparative methods include comparison of selected spectral variables, comprehensive methods that fully analyze underlying spectral features such as, but not limited to, discrimination function development using linear and non-linear combinations of spectral data, statistical model development and testing, genetic algorithms, as well as computationally ‘intelligent’ methods such as a neural net.

Based on results of the Standard Spectral Survey and Advanced Spectral Profile, the conditions are defined for molecular identification. The wavelength providing the most significant discriminatory signal is selected for molecular detection coupled to chromatographic methods. The spectral patterns of the molecules eluted from systems reiterate the discrimination of the aggregate preparations. The confirmation of this effect by comparison of multiple diseased and control samples provides significant scientific validation of the molecular marker of the discriminatory signals.

The molecular constituents that contribute to the discriminatory signal are then purified and structural identification by mass spectrometry is established.

In some cases, the native fluorescence of the target molecule will permit its direct detection in patient samples, however, in other cases, physiological conditions will prevent the detection of the target molecule based on intrinsic fluorescence in sample preparations. The invention may utilize an assay for the subject target molecules that employs an alternative fluorogenic molecule linked to a second entity that will specifically interact with the target molecule. This approach will validate the presence of the target molecule in the disease process being studied and lead to a family of specific and highly sensitive test procedures that will efficiently utilize a common platform instrument such as that described in U.S. Pat. No. 6,265,151.

The application of SMI is typically directed to medical applications where the need for specific molecular information is well recognized, but has many additional applications in pharmaceutical process control, food and beverage processing, and for environmental detection of toxic substances, for example. For drug processing and food and beverage manufacturing, the fluorescence assay will profoundly limit the scope of product recalls and reduce the economic impact of these events. Specific applications include but are not limited to: bacterial testing in meat and poultry, e.g., salmonella and E. coli, in process testing for beer and wine manufacture, fruit juice blending, and vegetable oil extraction. In each processed food product noted above, a standard color range is desirable for product release and progressive changes will occur throughout the production process that can be analyzed using SMI. The availability of real-time spectral data would be of great value in directly regulating final product quality.

The present invention is particularly well suited for the rapid detection of small fluorescent molecules and can be further enhanced to detect non-fluorescent molecules with other specific spectral signals as well. Several classes of highly toxic molecules that are considered potential terrorist weapons, such as the neurotoxins: VX gas and Sarin, contain specific chemical structures that produce distinct spectral signals. The invention can be optimized to rapidly detect these types of molecules in a system that would permit the screening of solid objects and liquids in a high through-put format such as mail testing, luggage surveillance, or random analysis of packaged fluids.

### EXAMPLES

The following examples are given to illustrate the scope of the present invention. Since these examples are given for illustrative purposes only, the present invention is not limited to the examples.

### Example 1

For intrinsic fluorescence (IF), biological samples (urine, blood, plasma, CSF, etc.) are prepared by a number of different procedures for parallel analysis. These may include, but are not limited to, simple dilution, organic solvent extraction or precipitation, acid extraction or precipitation, and PEG precipitation. Following preparation, the IF of several normal and diseased samples are surveyed using several wavelengths of excitation light, preferably 210 nm to 1.2 μm until a difference in spectral signals is detected.

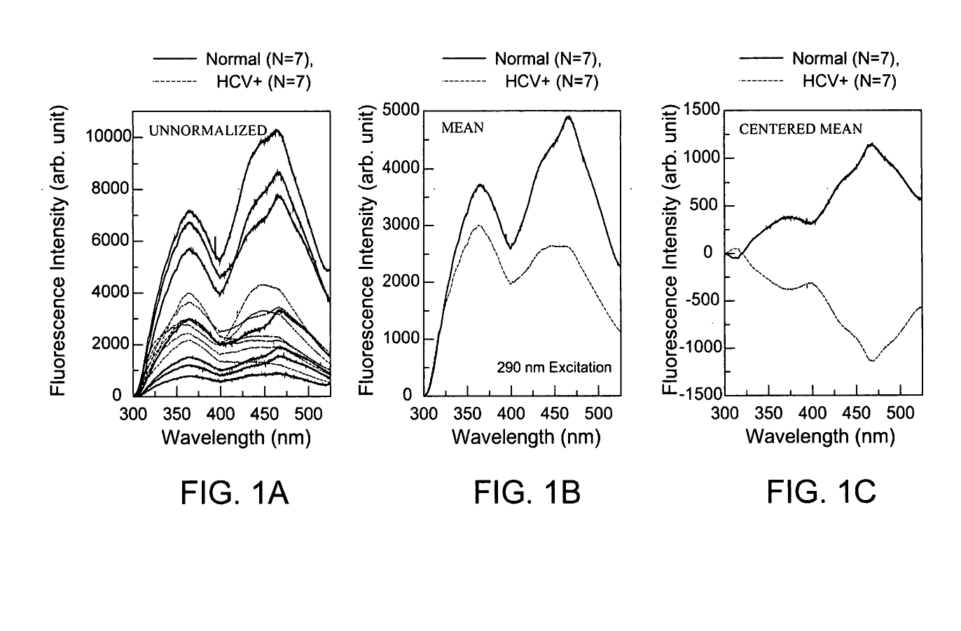

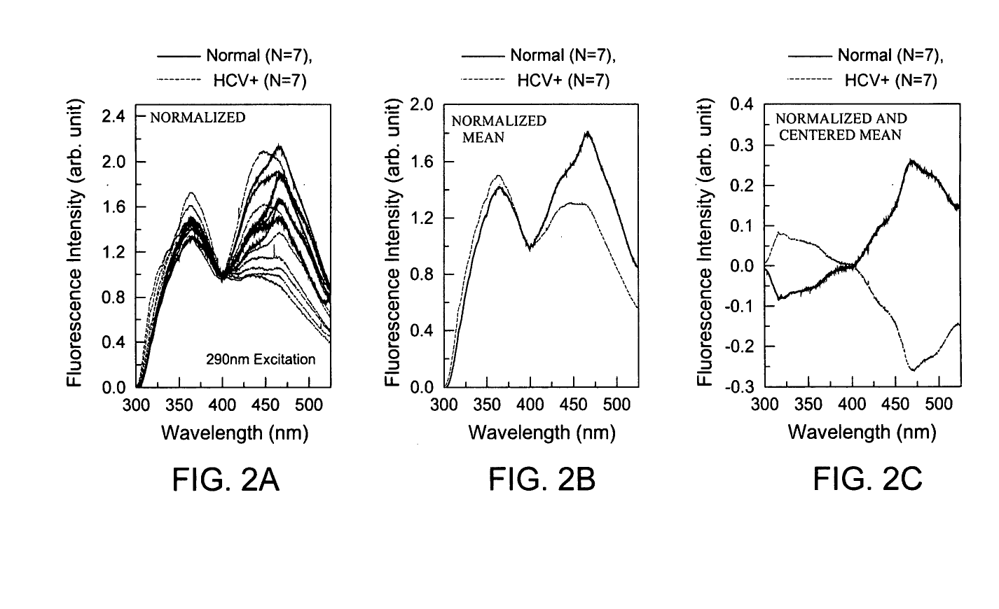

Using ACN/TFA extracted plasma samples from normal and HCV infected subjects and illumination with light of 290 nm, a consistent IF spectral difference is noted (FIG. 1a). Normalization of these spectra at a single wavelength provided a clearer view of the differences in spectral composition between normal and infected subjects (FIG. 2a). The mean spectra for the two groups (FIGS. 1b, 2b) further clarified the specific spectral differences between the two groups which was magnified by the subtraction of the mean for all the samples and is referred to as the centered mean (FIGS. 1c, 2c)

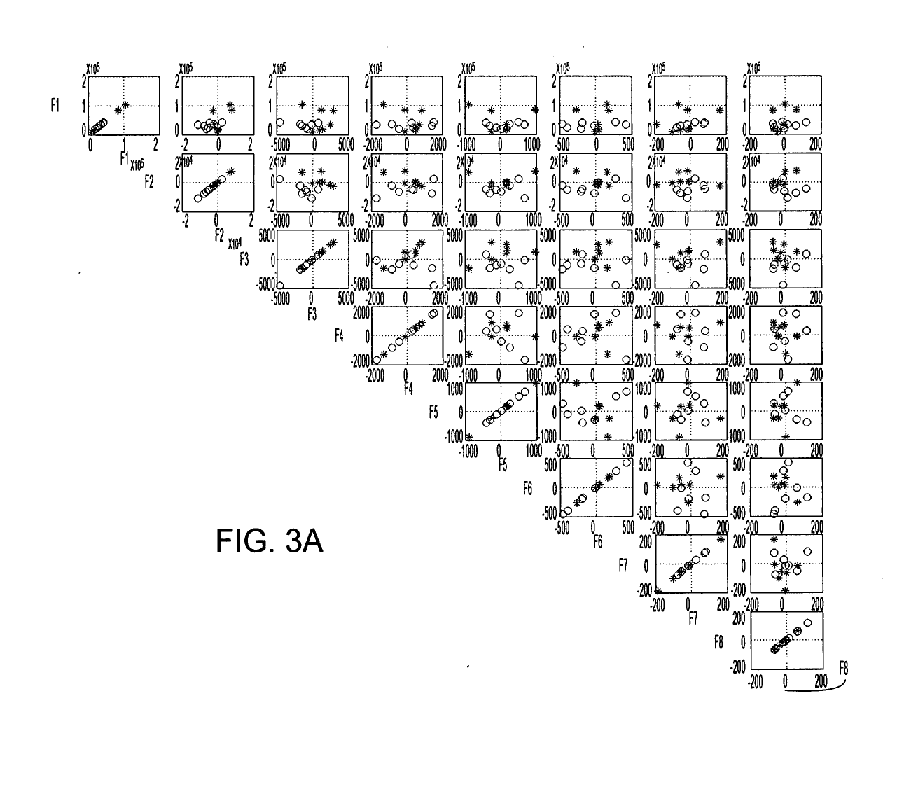

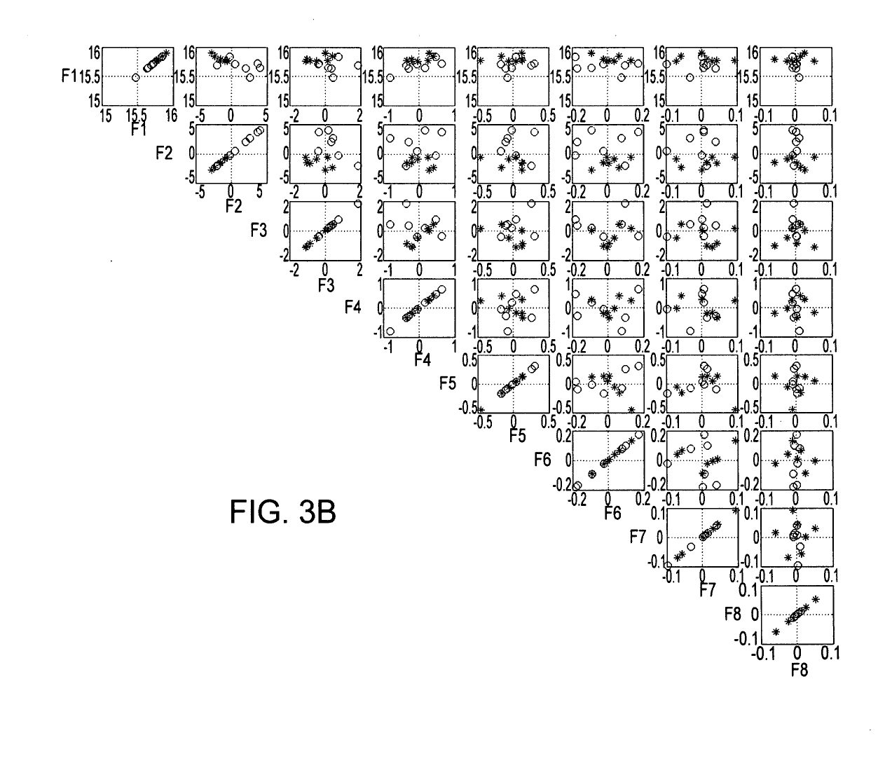

The full panel of spectra were analyzed by Principal Component Analysis (PCA) to identify spectral domains with the greatest level of variation. The initial analysis was limited to identification of the eight ‘factors’ carrying the greatest amount of signal variation. The individual spectra were then reclassified according to their infection status and projected onto the first eight principal component factors (FIG. 3). For both the normalized and unnormalized spectra, Factors 2 and 3 held a significant level of discriminatory information. For the normalized spectra (FIG. 3b), Factor 1 also held discriminatory information as indicated by the partial separation of the infected (O) and normal samples (*) along the diagonal axis of the F1×F1 plot (upper left).

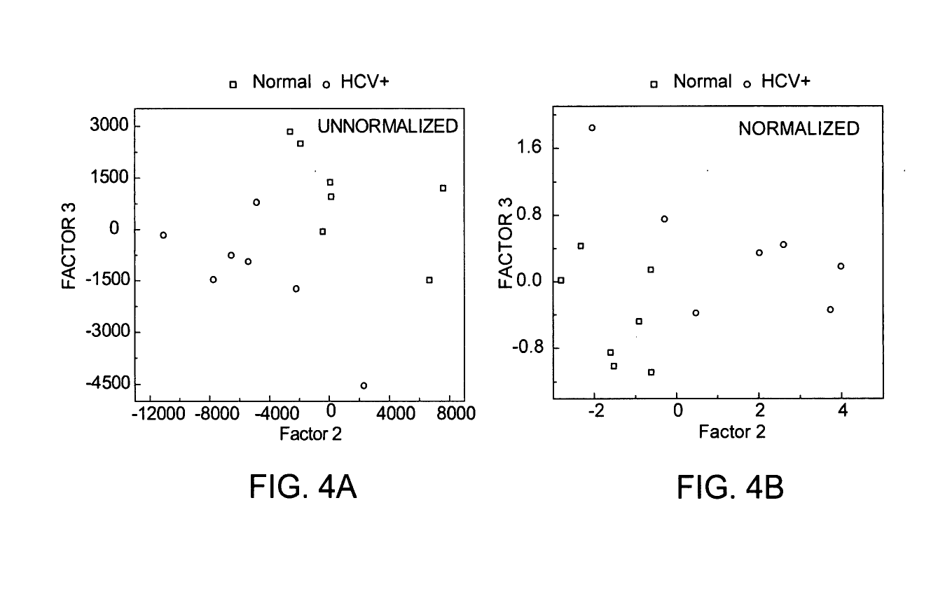

When analyzed as 2 dimensional combinations the combination of Factors 2 and Factor 3 provided full discrimination between the two groups in the unnormalized data set (FIG. 4a) and near full discrimination in the normalized data set (FIG. 4b). The individual components were subjected to univariate stepwise discriminate analysis which showed that, for the unnormalized data set, the differences between the infected and normal groups were statistically significant in Factor 2 (p<0.01) and Factor 3 (p<0.03). A multivariate analysis including all eight principal components further indicated the two groups to be significantly different (p<0.006).

A chromatography strategy is used by the invention that utilizes the key Spectral parameters identified to isolate the molecules carrying the discriminatory signal. For this purpose, extracted samples from several normal and HCV-infected individuals were subjected to reverse phase high pressure liquid chromatography (RP-HPLC). Elution of discriminatory molecules was monitored by simultaneous UV absorption (290 nm) and fluorescence (ex 290 nm/em 320 nm or ex 290 nm/em 440 nm).

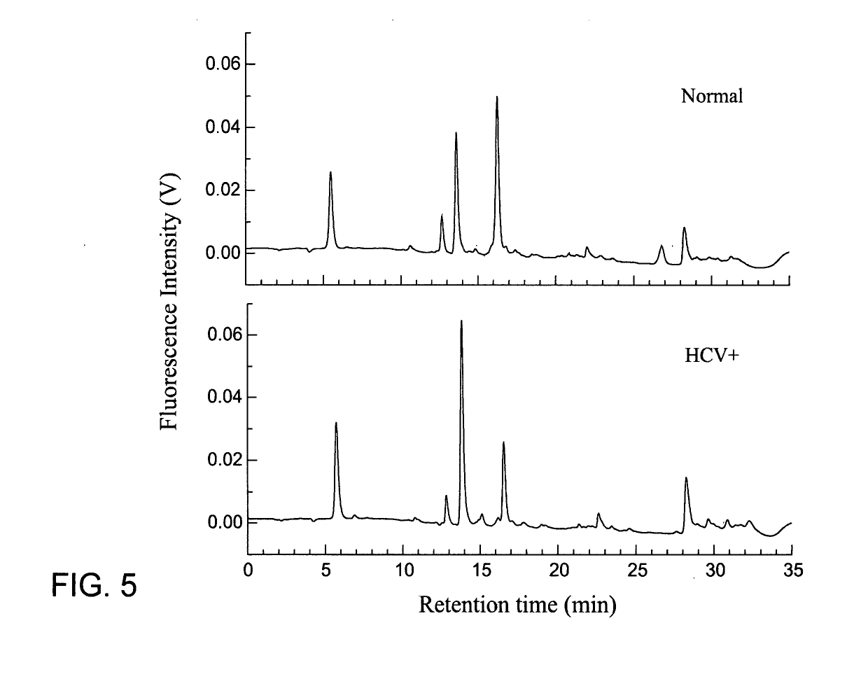

Differences in fluorescence (ex 290 nm/em 320 nm) elution profiles for normal and infected samples were notable. Typical chromatographs are presented in FIG. 5.

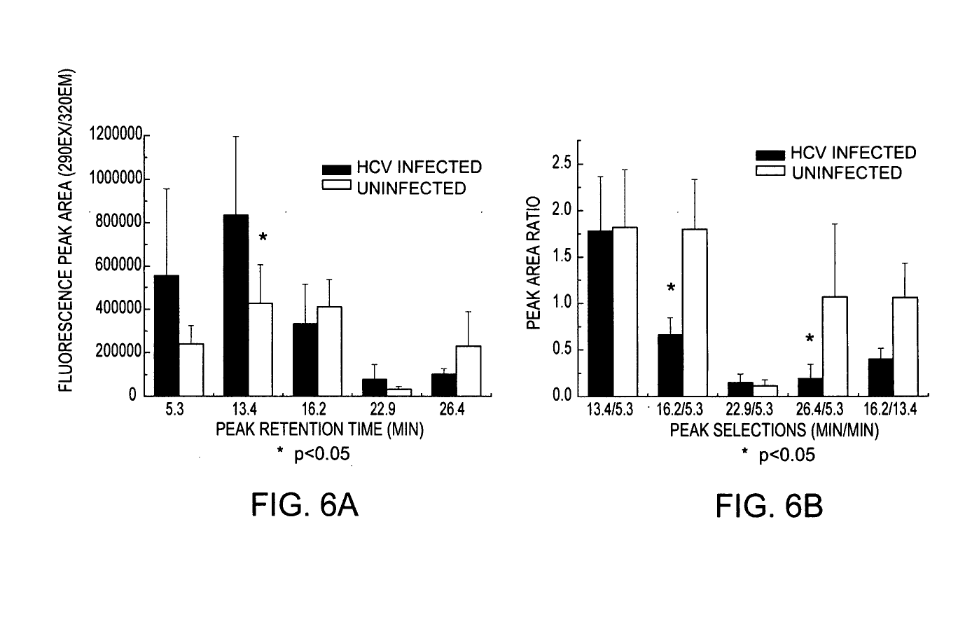

Using excitation light of a wavelength (290 nm) that provided discriminatory signals in the bulk extract and a detection wavelength (320 nm) where differential spectral signals were notable, molecular peaks eluted reproducibly at around 5.3, 13.8 and 16.2 minutes. Chromatographic processing of a panel of infected and normal samples provided results permitting the determination of the quantitative relationship of the amounts of each peak (peak area) with disease and statistical analysis of any correlations established. Quantitative increases in the 5.3 and 13.4 minute peaks were associated HCV infection (FIG. 6a). The increase in the 13.4 minute peak was statistically significant (p<0.05), however, careful inspection of the spectra suggested that the most profound differences between the two groups was more likely to be seen in the proportions of the major peaks which would normalize for any differences in amounts loaded for each analysis. When analyzed as peak area ratios (FIG. 6b), the ratio of the 16.2 and 5.3 minute peaks was significantly (p<0.05) reduced in the HCV positive group. Similarly, the ratio of the 16.2 and 13.4 minute peaks was significantly (p<0.05) reduced in HCV-infected samples.

Based on the statistical analysis of the peak ratios, it is concluded that the molecules represented by these peaks carry the discriminatory information established previously by the spectral profiling strategy. The combination of the two significant ratios may provide enhanced discriminatory power suggesting that the levels of all three molecules may be altered during HCV infection.

The material eluting from the RP-HPLC system noted above at 5.3, 13.4 and 16.2 minutes was collected and subjected to mass spectrometric (MS) analysis following rechromatography under similar conditions. Tandem MS revealed each chromatographic peak to contain several mass ions in the range of 250 to 2500 Daltons that represented a coherent set of fragment of 3 discrete molecular species, called SXI-18053, SXI-18134 and SXI-18162.

### Example 2

Spectra-Molecular Informatics

Elucidation of Hepatitis C virus Molecular Markers by Spectra-Molecular Surveillance

Standard Spectral Survey

Human plasma and serum samples from normal and HCV-infected patients were processed by several methods (e.g. simple dilution, ACN/TFA extraction), and scanned by standard spectroflorometric methods as well as exposed to excitation light of several selected wavelengths. The fluorescence spectra obtained from these studies revealed subtle differences between samples from infected and normal subjects that provided the foundation for more specific studies.

Advanced Spectral Profile

Based on the preliminary results noted above and specific biological knowledge relevant to the pathogenicity of the Hepatitis C virus, an alternative preparative method employing precipitation of plasma with polyethylene glycol (PEG) and additional wavelengths of excitation light were evaluated further in addition to the ACN/TFA extraction method. Such preparative methods are discussed in copending application Ser. No. 10/144,778, filed May 15, 2002, which is incorporated by reference.

A.

For samples extracted with ACN/TFA, excitation wavelengths of 260, 290, 320, 355, 380, 420, 580, 640 nm were evaluated in preliminary studies. Comprehensive studies focused on the assessment of fluorescence spectra derived from excitation light of 290, 320, 355 and 380 nm due to the higher level of fluorescence intensity and spectral complexity associated with these wavelengths.

Analysis of spectral data from sets of normal and HCV infected plasma donors revealed significant differences between these two groups in the fluorescence spectra obtained from 290 nm excitation, indicating the differential presence of one or more fluorescent molecules that could be detected with these specific conditions (i.e. 290 nm excitation, 300-500 nm emission). Identification of the ‘discriminatory spectral signal’ is a critical element of the Spectra-molecular Informatics strategy, as it provides the information regarding instrument settings needed to isolate the ‘discriminatory molecule(s)’.

B.

Both the supernatants and resuspended pellets of samples that were precipitated with PEG were illuminated with light of multiple wavelengths (320, 355, 380 μm). The samples derived from the PEG pellets (a lipoprotein rich fraction depleted of albumin) showed significant differences between the fluorescence spectra of the normal and infected groups under 355 and 380 nm illumination. These additional discriminatory signals provide additional information concerning the biochemical nature of the discriminatory molecule and an alternative approach to its purification that would rely on these spectral features.

Identification of HCV Discriminatory Molecules

Based on the results of the Advanced Spectral Profile, ACN/TFA extracted plasma samples were examined chromatographically in several solvent systems and using several chromatographic approaches, with fluorescence at 290 nm excitation as the detection criteria. Reverse phase HPLC, with a C-18 column and acetonitrile elution gradient separated multiple fluorescent peaks representing the molecular components of the mixture that offered the discriminatory spectral signal. Comparison of the chromatographic profiles between control and diseased samples revealed significant quantitative differences in specific peak areas between the two groups. Most notably, a significant increase in the area of a peaks eluting at approximately and 5.3 and 13.4 minutes was associated with HCV infection. A parallel decrease in the area of the peak eluting at 16.2 minutes provided a ratio (16.2 min/5.3 min) with substantial discriminatory power.

The material eluting from the RP-HPLC system noted above at 5.3, 13.4 and 16.2 minutes was collected and subjected to mass spectrometric (MS) analysis following rechromatography under similar conditions. Tandem MS revealed each chromatographic peak to contain several mass ions in the range of 250 to 2500 Daltons that represented a coherent set of fragment of 3 discrete molecular species, called SXI-18053, SXI-18134 and SXI-18162.

Development of Prototype Assays and Validation of HCV Discriminatory Markers While the identity of the discriminatory molecular species holds significant value, the relative importance of their contributions to the overall discriminatory signal offers additional information regarding their utility in disease diagnosis and treatment. To quantify the amount of each discriminatory molecule in HCV infected plasma samples, a biochemical assay is used based on the binding of the discriminatory molecule to a specific binding entity.

In the case of SXI-18053, the assay employs a fluorescently tagged version of SXI-18053 (SXI-18053-FL23) that binds with similar affinity to untagged SXI-18053 to the selected protein. In the absence of soluble SXI-18053, the protein binds the tagged molecule and can be immunoprecipitated or filtered to separate the complex from the free SXI-18053-FL23. In the presence of free SXI-18053, the tagged molecule is displaced in proportion to their relative amounts, and a reduced amount of tagged molecule is associated with the binding agent after separation. Either dissociated or enzyme associated signal can be measured to quantify the amount of SXI-18053 in a test sample.

The assay is a general competitive-binding assay that can alternatively employ an antibody, carbohydrate, nucleic acid or other molecule as the binding agent. The compound used to tag the discriminatory molecule for its specific analysis can include radioisotopes, fluorescent compounds, enzymes, avidin, biotin, and other detectable agents. For the assays described here, a common fluorescent tag (FL23) has been employed that is optimally excited and fluorescent in a spectra range distinct from the spectra of the subject molecules. These assays are extrinsic fluorescent systems that benefit from the use of a common instrument for their detection while employing target-specific reagents.

These assays are utilized to evaluate the levels of SXI-18053, SXI-18134 and SXI-18162 in plasma derived from normal and infected patients and provide quantitative confirmation of these molecules as valid independent markers of Hepatitis C infection. The relative levels of these molecules provide further information concerning biochemical pathways that are disrupted by HCV infection and would be important targets for therapeutic drug development.

According, it should be readily appreciated that the methods of the present invention has many practical applications. Additionally, although the preferred embodiments have been illustrated and described, it will be obvious to those skilled in the art that various modifications can be made without departing from the spirit and scope of this invention. Such modifications are to be considered as included in the following claims.

What is claimed is:

1. A method for identifying compounds associated with the presence of disease, comprising:
obtaining a plurality of fluorescence spectra, each spectrum being obtained from a respective biological sample having a known disease state;
identifying at least one spectral feature indicative of the presence of the disease; and
identifying at least one compound based upon the spectral feature.

2. The method of claim 1, wherein the identifying at least one compound step comprises subjecting at least a portion of a biological sample to a chromatographic separation.

3. A method for identifying compounds as in claim 1 wherein:
the fluorescence spectra is a product of intrinsic fluorescence of the sample.

4. A method for identifying compounds as in claim 1 wherein:
the identified compound is a particular molecule.

5. A method for identifying compounds as in claim 1 further comprising:
preparing the sample to selectively amplify the identified spectral feature.

6. A method for identifying compounds as in claim 5 wherein:
sample extracting methods are used to analyze a plasma sample.

7. A method for identifying compounds as in claim 1 wherein:
the biological sample is selected from the group consisting of plasma, serum, urine and spinal fluid.

8. A method for identifying compounds as in claim 1 wherein:
the fluorescent spectra is obtained from an excitation source having a wavelength of between 190 and 1200 nanometers.

9. A method for identifying compounds as in claim 8 wherein:
the wavelength of the excitation source is between about 260 and 640 nanometers.

10. A method for identifying compounds as in claim 9 wherein:
the wavelength of the excitation source is about 290 nanometers.

11. A method for identifying compounds as in claim 1 wherein:
multivariate statistical methods are used to identify the spectral feature.

12. A method for identifying compounds as in claim 11 wherein:
the multivariate statistical methods used are Principal Component Analysis and Independent Component Analysis.

13. A method for identifying unknown markers associated with the presence of a predetermined characteristic comprising:
creating a first spectral survey of a sample with a predetermined characteristic and a sample without such characteristic based on predetermined preparative methods applied to such samples and predetermined excitation wavelengths used to irradiate such samples;
creating a second spectral survey wherein further spectral resolution of the samples is acquired;
subjecting the first and second surveys to analytical processing wherein a sample with the predetermined characteristic is differentiated from a sample without the characteristic based on its emission spectrum;
isolating the emission wavelength which demonstrates the greatest differentiation; and
determining the marker that demonstrates the wavelength causing the greatest differentiation.

14. A method for identifying markers associated with the presence of a predetermined characteristic comprising:
accumulating spectral data comprising intrinsic fluorescence from a sample known to have a predetermined characteristic;
identifying a spectral feature which is indicative of the predetermined characteristic;
identifying a marker associated with the spectral feature.

15. A method for identifying molecules associated with the presence of disease comprising:
obtaining a biological sample having a known disease state;
preparing the sample to amplify an intrinsic fluorescent signal from different molecules;
analyzing the intrinsic fluorescence over a range of excitation and emission wavelengths;
identifying a spectral feature that provides disease discrimination;
identifying a molecule that produces the spectral feature.

16. A method for the identification of disease specific molecules comprising:
obtaining a sample having a known disease state;
analyzing the intrinsic spectral features of the sample;
identifying a spectral feature that provides specific disease discrimination;
identifying a molecule that produces the spectral feature.

17. A method for identifying disease related discrimination spectral signals comprising:
obtaining multidimensional spectral data from a sample having a known disease state;
identifying the spectral data that indicates the presence of the disease;
identifying compounds that express the identified spectral data.

18. A method as in claim 17 wherein:
the multidimensional spectral data includes intrinsic fluorescence.

19. A method as in claim 17 wherein:
the identified data indicates the presence of a predetermined disease.

20. A method as in claim 17 further comprising:
utilizing data reduction methods to amplify the spectral data.
